(19) United States
(12) Patent Application Publication
(10) Pub. No.: US 2008/0103512 A1
(43) Pub. Date: May 1, 2008

(54) CLAMPING SYSTEM AND METHOD FOR FUSING VERTEBRAL ELEMENTS IN A SPINE

(75) Inventor: Nicholas Gately, Lambertville, NJ (US)

Correspondence Address: NIXON & VANDERHYE, PC, 901 NORTH GLEBE ROAD, 11TH FLOOR, ARLINGTON, VA, 22203, US

(73) Assignee: G&L CONSULTING, LLC, New York, NY (US)

(21) Appl. No.: 11/876,933

(22) Filed: Oct. 23, 2007

Related U.S. Application Data
(60) Provisional application No. 60/862,511, filed on Oct. 23, 2006.

(51) Int. Cl. A61B 17/08; A61B 17/58

(52) U.S. Cl. 606/151; 606/61

(57) ABSTRACT

A facet joint clamp to fix spine facet joints in a spine of a mammalian patent, the clamp including: a first plate and second plate each including a jaw section and a base, wherein the base is at an acute angle with respect to the jaw section, and the jaw section has an inside surface adapted to grasp the spine; a hinge in the base section of the first and second plates and forming a pivot joint between the plates and an adjustment mechanism to controllably pivot the first plate with respect to the second plate.

### BACKGROUND OF THE INVENTION

This application claims the benefit of U.S. Provisional Patent Application Ser. No. 60/862,511, filed Oct. 23, 2006, the entirety of which is incorporated by reference.

The present invention relates to surgical devices and methods to clamp a spinal facet joint and instruments for steering and applying clamps to the spinal facet joint.

Traumatic, inflammatory, metabolic, and degenerative disorders of the spine can produce debilitating pain. A common surgical interventions today is arthrodesis, e.g., spinal fusion. Spinal fusion surgery generally requires a surgeon to access the spine and apply treatment devices, e.g., screws, rods, bone grafts and artificial discs, that effectively fuse adjacent vertebra.

A traditional surgical approach to the spine is through the back of a patient. The surgeon cuts a mid-line incision in the back that is approximately three inches to six inches long to access the spine. A bone graft is obtained from the iliac crest of the pelvis and the harvested bone graft is laid out in the posterolateral portion of the spine. This posterolateral portion lies on the outside of the spine and is a very vascular area. The rich vascular area provides the bone graft with blood to supply nutrients for bone growth and fusion.

A small extension of the vertebral body in this area (transverse process) is a bone that serves as a muscle attachment site. The muscles that attach to the transverse processes are elevated to create a bed on which to lay the bone graft. The back muscles are then laid back over the bone graft, creating tension to hold the bone graft in place.

With supplemental fixation (rods and pedicle screws) typical successful fusion rates are 70%. Because the vertical compression loads of the spine do not pass directly through the axis of the rod, rod and screw systems cannot resist these loads without considerable flexing, reducing the incidence of fusion. Methods to improve fusion rates led to better stabilization of the fusion with the development of interbody cages and a three point (two rods and an interbody device) construct able to resist flexure.

Rod and screw systems are generally not suited to minimally invasive surgical (MIS) techniques because the rods span vertebral levels and must be forced through muscle tissue between the vertebrae, causing significant tissue trauma. There are many MIS rod and screw systems, but most are unsatisfactory and some only span one level. Accordingly, there is a long felt need for an improved spine fusion system and method.

### SUMMARY OF THE INVENTION

A facet joint clamp has been developed to fix spine facet joints in a spine of a mammalian patent, the clamp including: a first plate and second plate each including a jaw section and a base, wherein the base is at an acute angle with respect to the jaw section, and the jaw section has an inside surface adapted to grasp the spine; a hinge in the base section of the first and second plates and forming a pivot joint between the plates and an adjustment mechanism to controllably pivot the first plate with respect to the second plate.

A method has been developed to position a facet joint clamp on a spine of a mammalian patent, the method comprising: coupling the facet joint clamp to a steering instrument, wherein the facet joint clamp includes a first plate and second plate hinged together and each including a jaw section and a base, wherein at least one the bases is releasably coupled to the steering instrument; using the steering instrument to insert the clamp into a patient and seat the jaw sections of the first plate and the second plate against the spine; with the jaw sections seated on the spine, causing the first plate and second plate to pivot with respect to each other to clamp the jaw sections on the spine and fix the clamp to the spine, and releasing the steering instrument from the base.

### BRIEF DESCRIPTION OF THE DRAWINGS

FIG. 1 shows a pair of facet joint clamps attached to the spine to fix bones being fused.

FIG. 2 shows a facet joint clamp attached to the spine to fix bones being fused.

FIGS. 3A to 3E are various views of a facet clamp with angled adjustment screws that adjustably close the clamp onto a spine and having an attachment for steering the clamp to the spine during surgery. FIG. 3A is a side view of the clamp with the jaws open. FIG. 3B is a front view of the clamp showing the jaws open. FIG. 3C is a side, cross-sectional view of the clamp with the jaws open. FIG. 3D is a front, side perspective view of the clamp. FIG. 3E is a side view of the clamp with the jaws closed (such as would be used to fix to facet bones).

FIGS. 4A and 4B include an enlarged perspective view (FIG. 4A) of the facet clamp to show the adjustable screws and an exploded view (FIG. 4B) of the clamp.

FIGS. 5A to 5C include various views of another embodiment of a facet clamp with an angled adjustment screw that adjustably closes the clamp onto a spine. FIG. 5A is a side view of the clamp with the jaws open. FIG. 5B is a front view of the clamp showing the jaws open. FIG. 5C is a side, cross-sectional view of the clamp with the jaws open. FIG. 5D is an exploded view of the clamp.

FIG. 6A is a rear, side perspective view of a plate of a clamp with a steering attachment fixed to the steering post on the clamp.

FIG. 6B is a front perspective view of the steering attachment and shows the attachment mechanism.

FIG. 7 is a perspective view of a steering device with an attached clamp.

### DETAILED DESCRIPTION OF THE INVENTION

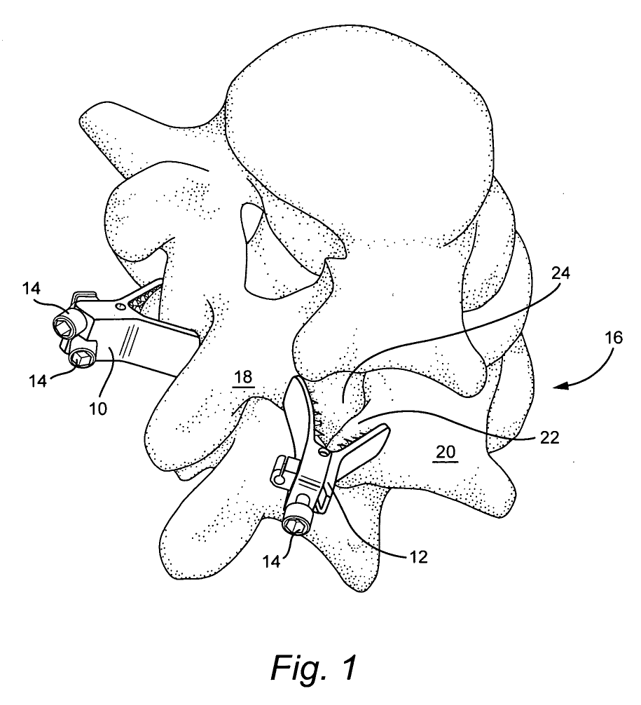

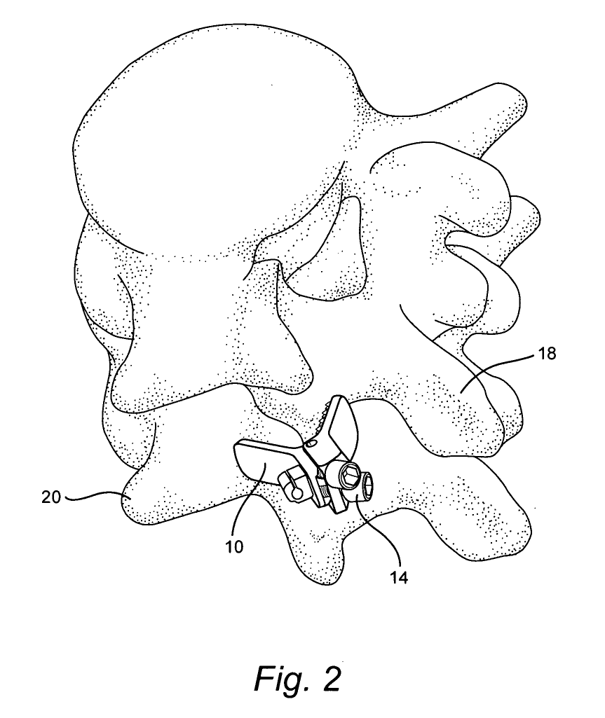

FIG. 1 shows a pair of facet joint clamps 10, 12 attached to the spine to fix bones being fused. Similarly, FIG. 2 shows a facet joint clamp attached to the spin to fix bones being fused.

The jaws of the clamps pivot under the control of adjustment screws 14. The clamps may be surgically inserted into a patient and to the spine 16 with the jaws held wide-open. The clamps 10 when applied to the spine may hold together adjacent upper and lower vertebra 18, 20. For example, the clamps may grasp opposing facet joint bones 22, 24.

The surgeon steers the clamps 10 to the spine and seats the clamp on the spine with a steering device. The jaws may be seated such that they are adjacent outer surfaces of opposing facet joint bones 22, 24. Once the clamp is properly seated, the surgeon closes the jaws of the clamp by turning the adjustment screws 14. The screw force the jaws to close and securely grasp the bones of the spine. The number of turns applied to the adjustment screw and the torque applied to the screws determines the force applied by the clamp jaws to hold the spine bones together.

The clamps 10 when secured to the spine are an interbody device that provides a rigid and stable three point construct for holding together the spine facet joints. Multiple clamps can be applied to the spine to provide the desired degree of rigidity to the spine. The clamps may be used to achieved fusing of the facet joints. The clamps may be used instead of adding two vertical rods and attaching them to the spine by pedicle screws. A facet joint clamp 10 or plurality of clamps 10, 12 may be used to stabilize the facet joint and aid facet joint fusion.

The facet joint clamp(s) 10, 12 is small enough to be suitable for minimally invasive techniques. Once the clamp is positioned on a spine, two halves of the hinged clamp are closed together by an adjusting screw. The inside faces of the facet joint clamp have sharp teeth and serrations that grip the inferior and superior vertebra close to the facet joint and immobilize the joint. Another embodiment of the clamp involves a sharp, perhaps hollow pin on one clamp half that is driven through the facet joint, to immobilize the joint.

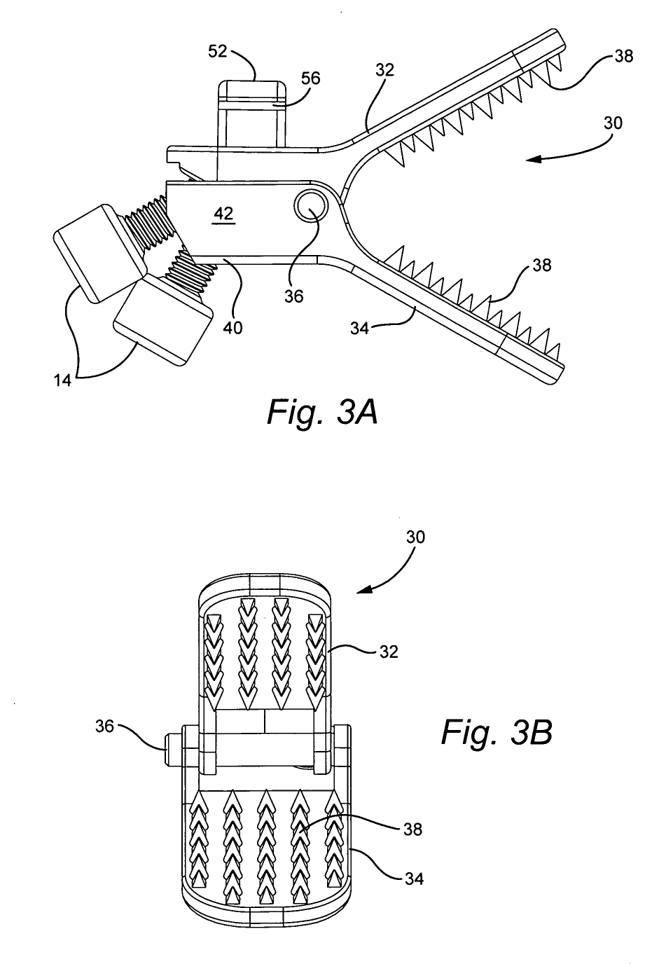

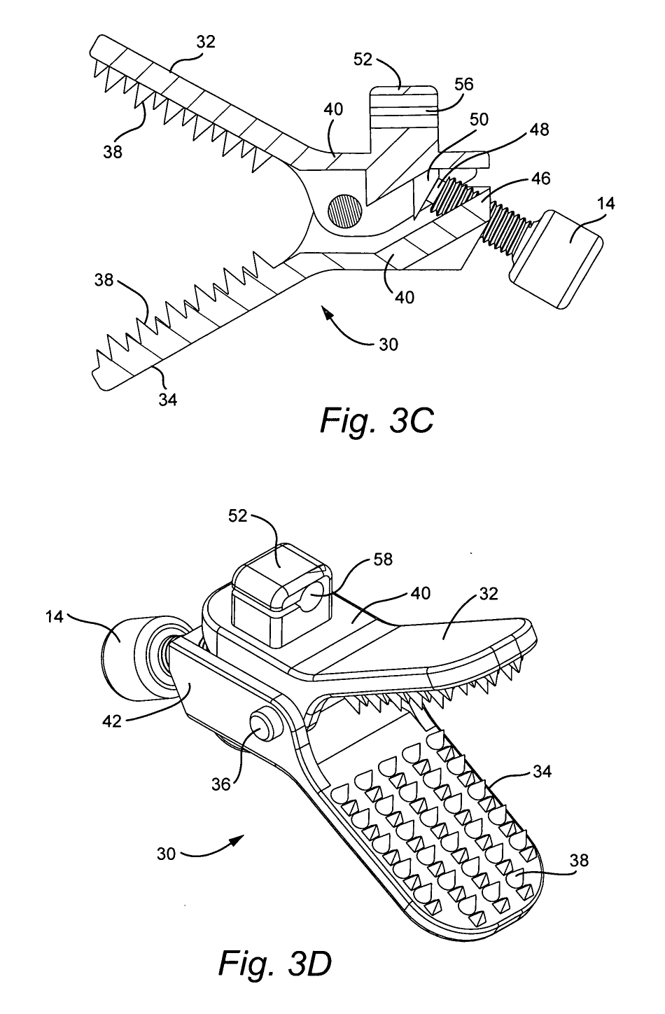

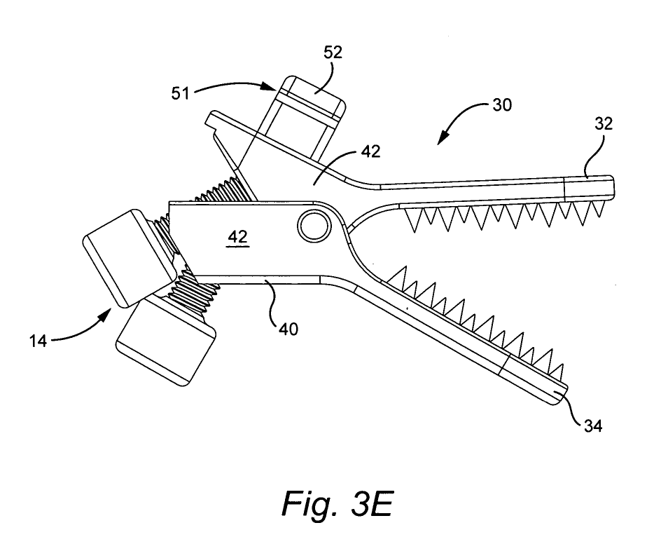

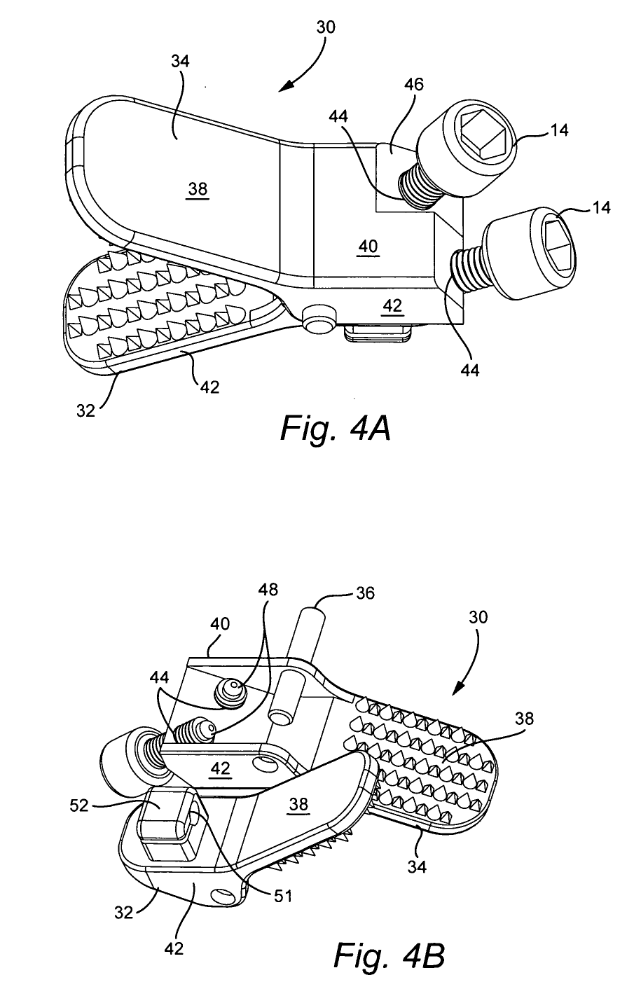

FIGS. 3A to 3E are various views of a facet clamp 30. The clamp 30 is suitable for use as the clamps 10, 12 shown in FIGS. 1 and 2. FIGS. 4A and 4B are an enlarged perspective view (FIG. 4A) of the facet clamp 30 to show the adjustable screws and an exploded view (FIG. 4B) of the clamp 30.

The clamp 30 comprises a first plate 32, an opposing plates 34 and a hinge 36 about which the plates pivot. Each plate comprises a jaw section 38 that is generally a planer metal or rigid plastic structure having an inside surface that is dimpled, serrated, knurled, roughened, populated with small needles or micro-needles or otherwise treated to grasp the surface of a spine.

The plates 32, 34 also include a base 40 that is at an angle, e.g., between 5 degrees to 20 degrees, with respect to the jaw section. The base may include a rigid sidewall 42 that provides structural support for the plate and apertures for the hinge pin 36 and threaded apertures for one or a pair of adjustment screws 14. The sidewalls and all edge surfaces of the clamp (with the possible exception of the inner surface of the jaw) may be smoothed and rounded.

Each plate includes one or more angle adjustment set screws 14 that adjustably close the clamp onto a spine. As is best shown in FIGS. 4A and 4B, the screws extend through threaded apertures 44 in the base 40 of a plate. The aperture 44 may in the rear sidewall 44. A first adjustment screw 14 may extend substantially laterally out from the end of the base and a second screw may extend at an angle, e.g. 45 degrees, with respect to the base. A facet 46 at an angle, e.g., 45 degrees, in the base may include a threaded aperture 44 for the angled adjustment screws.

Both set screws 14 can be turned to close the jaws together and secure the clamp to the spine. The turning of the adjustment screw may be by a turning device, e.g., hexed-end screw driver, operated by the surgeon. As the screw is turned, the screw advances into (or out of depending on the turning direction) the aperture 44. As the screw moves inward, the nose 48 of the screw abuts against an angled post 50 on an inside surface of the base 40 of the opposite plate.

The displacement of the screw nose causes the plates to pivot about the hinge and close onto the spine. The nose of the screw may be rotatably attached to the post 50 so that turning the screw in a first direction causes the jaws to close onto a spine and turning the screw in the opposite direction opens the jaws.

By providing two adjustment screws 14 at different angles, a surgeon can select the screw that is easiest to access with the turning device. Once the clamp has been positioned onto the spine, it may be difficult for the surgeon to access one of the screws. Having a second adjustment screw allows the surgeon to fix the clamp to the spine even if one screw is not accessible.

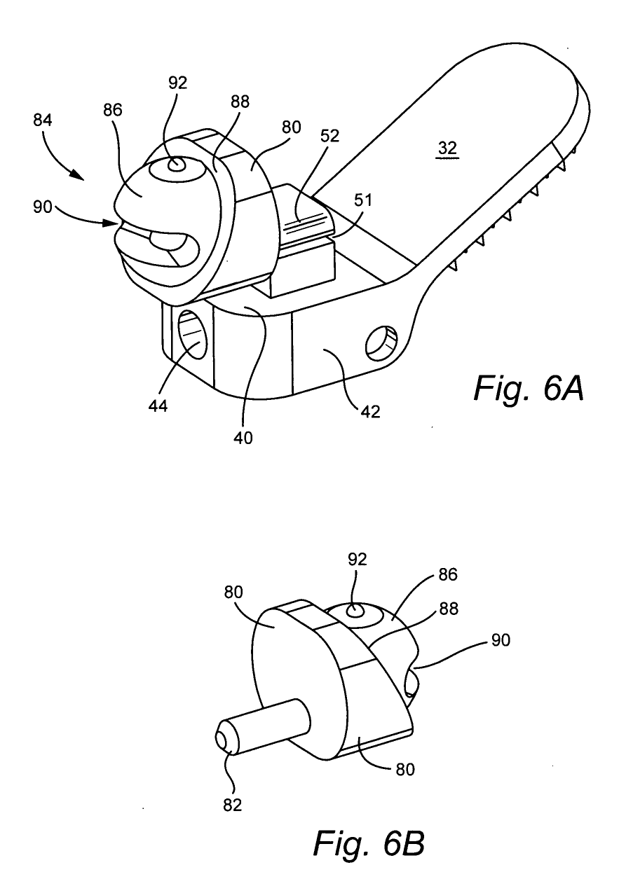

At least one plate in the clamp 30 may have an steering post 52 for steering the clamp to the spine during surgery. The steering post 52 may be a rectangular block on an outer surface of a base 40 of one of the plates. The post may include a keyhole shaped aperture 51 having an entrance slot 56 and a cylindrical aperture 58. A steering attachment (80, 82—FIG. 6) has an engagement device, e.g., a shaft to frictionally fit the keyhole aperture 51, or a plate which fits into the slot 56, or a hook that slides into the slot and seats in the cylindrical aperture. When the engagement mechanism is releasably attached to the aperture 51 and steering post 52, a steering device is a capable of orienting the facet clamp to an anatomically correct position on the superior and inferior vertebra. After attaching, closing and attaching the clamp to the vertebra so that the clamp is rigidly attached, the surgeon may remove the insertion instrument and steering attachment as one piece with a sharp pull and overcoming the frictional force between the keyhole aperture (56) and the shaft (82).

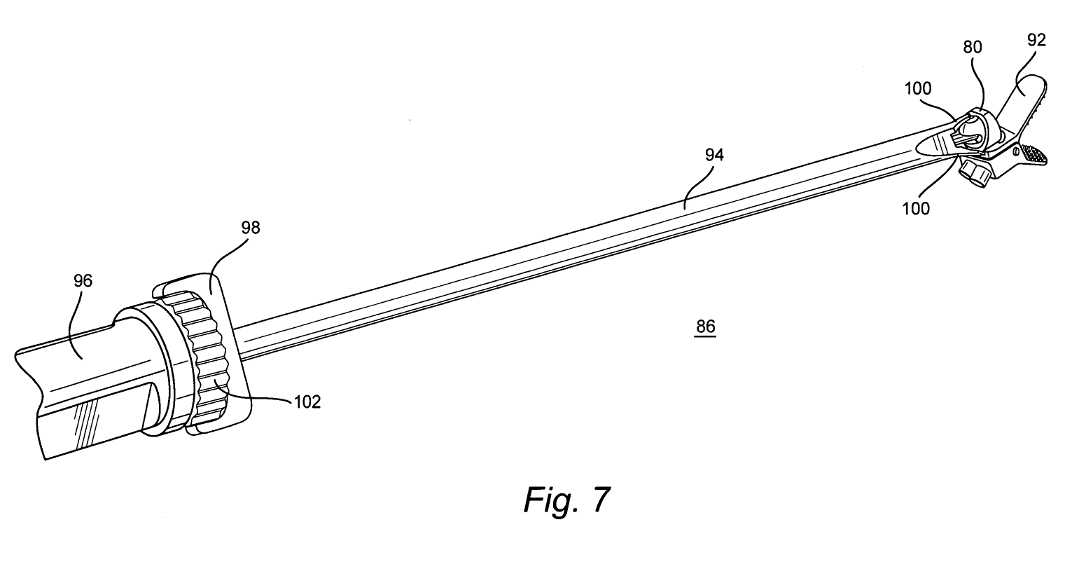

The steering attachment (80, 82 FIG. 6B) may also be fixed permanently to the facet clamp post (52, FIG. 6A) and the facet clamp released from the insertion instrument shown in FIG. 7 by rotating the handle (96, FIG. 7) disengaging the cylindrical pin 92, and so releasing the facet clamp.

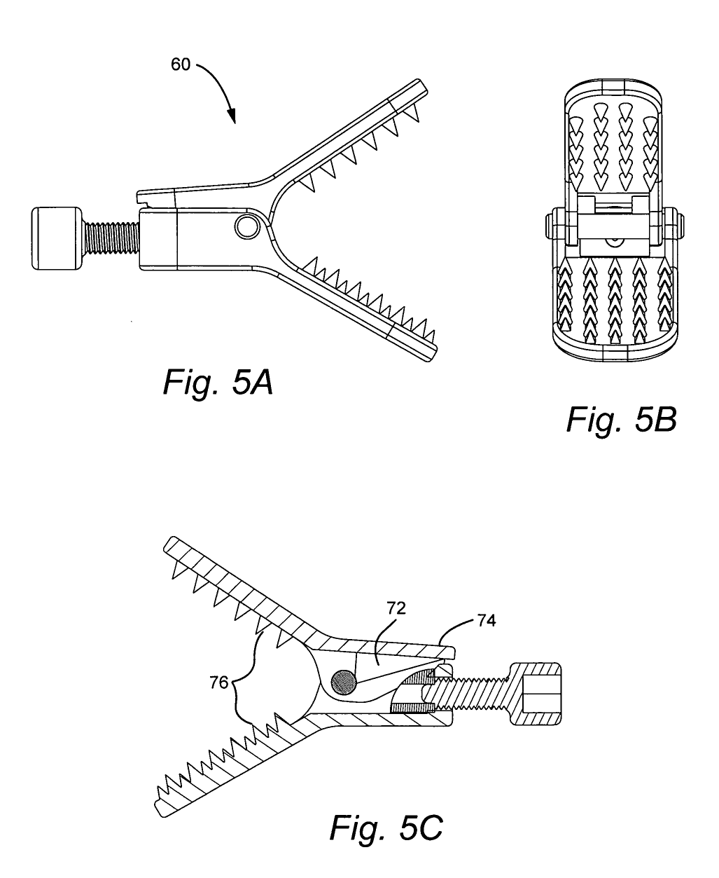

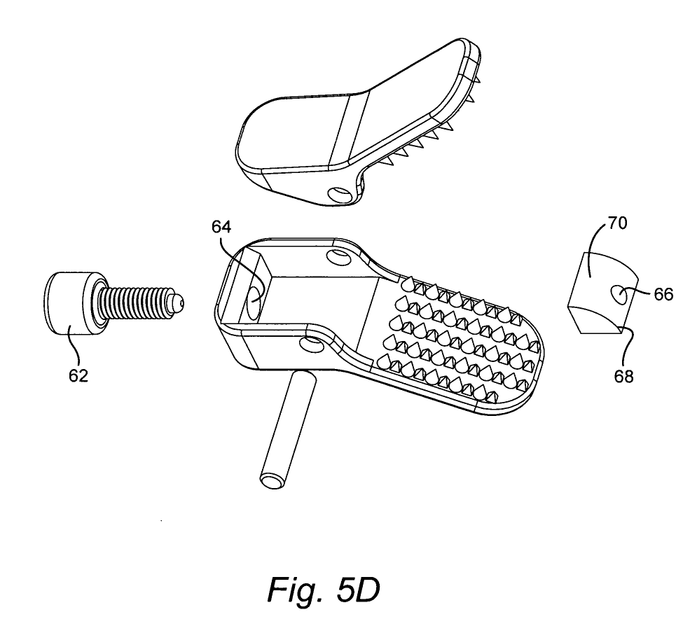

FIGS. 5A to 5D includes various views of another embodiment of a facet clamp 60 with an angled adjustment screw 62 that adjustably closes the clamp onto a spine. The clamp is similar in most respects to the clamp shown in FIGS. 3 and 4, except the that clamp 60 has a single adjustment screw 62. The screw fits through an aperture 64 (which may be un-threaded) and engages a threaded cylinder 66 of a slider block 68. As the screw turns, it moves the block 68 either forward or aft depending on the turn direction. A curved front surface 70 of the slider block 68 slides against a ramp surface of a post 72 on an inside surface of the base 74 of the opposite plate. The movement of the slider across the ramped post causes the jaws 76 to close together and grasp the facet joint bones of a spine.

FIG. 6A is a rear, side perspective view of a plate 32 of a clamp (see FIG. 3) with a steering attachment 80 fixed to the steering post 52 on the clamp. FIG. 6B is a front perspective view of the steering attachment and shows the attachment mechanism 82, which may be a key device that fits into the keyhole shaped aperture 51 of the post 52.

The steering attachment may have a face 84 (opposite to the attachment mechanism) that engages a steering instrument 86 (FIG. 7). The face 84 includes an hemispherical engagement 86 and an angled surface 88 extending around the hemispherical engagement. The hemispherical engagement provides a mount for a distal end of the steering instrument. The hemispherical engagement 86 comprises a slot 90 extending partially around the mid-line of the hemispherical engagement and provides a window into the hollow portion of the engagement. Within the hollow portion is a pin 92 that is rotatably mounted in the hemispherical engagement. The pin may extend through the center axis of the hemispherical engagement. The pin is attached to the distal end of the steering instrument and allows the end to turn and to move angularly from side to side within the slot 90.

The angled surface 88 is angled with respect to the shaft of the steering device. The angle surface 88 is a cam surface against which slides the front surfaces of cam wings rotatably attached to the shaft of the steering instrument.

FIG. 7 shows the steering instrument 86 attached to a clamp 92. The instrument includes a shaft 94 and a coaxial handle 96. The shaft includes an inner rod with a tip that is pivotably attached to the steering attachment 80. The tip may be threaded and engages a threaded aperture in a side of the cylindrical pin 92. The rod is fixed to the handle 96. Using the handle and rod, the clamp 92 can be guided to the spine by the surgeon.

The steering instrument is capable of moving the clamp 92 with respect to the rod. In particular, the instrument can move the clamp from side to side (a yaw movement) and rotate the clamp about the axis of the shaft. Rotation is provided by rotating the handle to rotate the shaft.

The shaft also includes a hollow tube coaxial with the rod. The distal end of the tube includes wing cams 100 that engage the cam surface 88 and fit around the hemispherical engagement 86. Yaw movement of the clamp is controlled by the a knob 98 on the tube of the shaft that rotates the cam wings 100 about the hemispherical engagement. The rotation of the knob, slides the front edges of the cam wings 100 across the cam surface 88 and forces the clamp 92 to move from side to side with respect to the axis of the instrument. As the clamp moves from side to side, the tip of the rod moves back in forth in the slot 90 of the hemispherical engagement 88.

The knob 98 at the handle 96 of the steering device enables a surgeon to rotate the cam wings 100 and thereby adjust the angle between the clamp and the axis of the instrument. Pivoting the clamp allows a surgeon greater movement in positioning the clamp onto the spine.

A locking knob 102 may be tightened to bind and lock the knob 98 effectively locking the clamp with respect to the shaft 94 of the steering instrument. When locked, axial force and torque can be applied to the handle 96 to advance the clamp into the spinal space and position the clamp adjacent the facet joint bones. Turning the locking knob 102 releases the knob 98 so that the surgeon can pivot the clamp into proper position on the spine.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A facet joint clamp to fix spine facet joints in a spine of a mammalian patent, the clamp comprising:
a first plate and second plate each including a jaw section and a base, wherein the base is at an acute angle with respect to the jaw section, and said jaw section has an inside surface adapted to grasp the spine;
a hinge in the base section of the first and second plates and forming a pivot joint between the plates and an adjustment mechanism to controllably pivot the first plate with respect to the second plate.

2. The facet joint clamp of claim 1 wherein the adjustment mechanism includes at least two adjustment screws each separately capable of pivoting the first plate with respect to the second plate and the first screw projected outward at a different angle than the second screw.

3. The facet joint clamp of claim 1 wherein the inside surface of the jaw section of each of the first and second plates includes at least one of dimpled, serrated, knurled, roughened and populated with small needles or micro-needles.

4. The facet joint clamp of claim 1 wherein the base of at least one of the first and second plates includes a steering post, wherein the post includes a coupling to releasably receive steering tool used to position the clamp on the spine.

5. A method to position a facet joint clamp on a spine of a mammalian patent, the method comprising:
coupling the facet joint clamp to a steering instrument, wherein the facet joint clamp includes a first plate and second plate hinged together and each including a jaw section and a base, wherein at least one the bases is releasably coupled to the steering instrument;
using the steering instrument to insert the clamp into a patient and seat the jaw sections of the first plate and the second plate against the spine;
with the jaw sections seated on the spine, causing the first plate and second plate to pivot with respect to each other to clamp the jaw sections on the spine and fix the clamp to the spine, and
releasing the steering instrument from the base.

6. The method of claim 5 wherein the steering instrument is released after fixing the clamp to the spine.

7. The method of claim 5 wherein the first plate and second plate pivot by turning an adjustment screw in a base of the first plate or second plate, wherein turning the adjustment screw applies a pivoting force to cause the jaw sections to pivot towards each other.

8. The method of claim 5 wherein using the steering instrument includes pivoting the clamp about a distal end of the instrument by actuating a handle of the instrument.

9. The method of claim 5 wherein the jaw section of the first plate is seated on a first vertebra and the jaw section of the second plate is seated on a second vertebra, wherein the second vertebra is lower than the first vertebra.

10. The method of claim 9 further comprising fusing the first and second vertebra by the fixation of the clamp to the spine.
